(19) United States
(12) Patent Application Publication
(10) Pub. No.: US 2026/0245652 A1
(43) Pub. Date: Aug. 20, 2026

(54) MECHANISTIC CHROMATIN MODELING WITH ITERATIVE OPTIMIZATION, NUCLEAR ENVELOPE INTERACTIONS, AND EDITABLE EPIGENETIC COMPOSITION

(71) Applicant: Altos Labs, Inc., Redwood City, CA (US)

(72) Inventors: Erik NAVARRO, Redwood City, CA (US); William POOLE, San Francisco, CA (US); Simone BIANCO, San Francisco, CA (US); Ryan SPANGLER, Seattle, WA (US)

(21) Appl. No.: 19/642,522

(22) Filed: Apr. 8, 2026

Related U.S. Application Data
(63) Continuation of application No. PCT/US2024/050764, filed on Oct. 10, 2024.
(60) Provisional application No. 63/589,281, filed on Oct. 10, 2023.

(51) Int. Cl. G16B 15/10; G01N 33/68; G16B 20/00

(52) U.S. Cl. CPC G16B 15/10; G01N 33/6875; G16B 20/00

(57) ABSTRACT

This disclosure relates to methods and systems for determining epigenetic states resulting from epigenetic modifying processes, particularly by use of molecules dynamics and models of epigenetic modifying processes.

### CROSS-REFERENCE OF RELATED APPLICATIONS

This application is a continuation of International Patent Application titled “MECHANISTIC CHROMATIN MODELING WITH ITERATIVE OPTIMIZATION, NUCLEAR ENVELOPE INTERACTIONS, AND EDITABLE EPIGENETIC COMPOSITION”, serial number PCT/US2024/050764, filed Oct. 10, 2024, which claims priority benefit of U.S. Provisional patent application titled “MECHANISTIC CHROMATIN MODELING WITH ITERATIVE OPTIMIZATION, NUCLEAR ENVELOPE INTERACTIONS, AND EDITABLE EPIGENETIC COMPOSITION”, Ser. No. 63/589,281, filed Oct. 10, 2023. The subject matter of these related applications is hereby incorporated herein by reference.

### BACKGROUND

Understanding the structure of chromatin is of paramount importance when it comes to human health. Chromatin is the dynamic and complex three-dimensional assembly of DNA and proteins found within the nucleus of eukaryotic cells. This intricate organization plays a pivotal role in regulating gene expression, DNA replication, and DNA repair, thus impacting various critical cellular processes.

Comprehending chromatin structure is crucial for deciphering gene regulation. Chromatin can exist in two main states: euchromatin, which is loosely packed and allows for gene transcription, and heterochromatin, which is tightly packed and represses gene expression. Dysregulation of these states can lead to various diseases, including cancer. An in-depth understanding of chromatin structure can facilitate the development of targeted therapies that modulate gene expression, potentially providing treatments for conditions where gene regulation plays a pivotal role.

Furthermore, chromatin structure is integral to DNA replication and repair mechanisms. During replication, the chromatin must be efficiently unwound and then faithfully reassembled to maintain genetic stability. Any errors or disruptions in this process can lead to mutations and genomic instability, which are often associated with a range of diseases, including neurodegenerative disorders and autoimmune conditions.

Chromatin structure is closely linked to epigenetic modifications. These modifications, such as DNA methylation and histone acetylation, can have profound effects on gene expression and are implicated in cardiovascular diseases, diabetes, and mental health disorders. Understanding how epigenetic modifications of chromatin interact can provide insights into disease mechanisms and potential therapeutic targets.

In summary, the structure of chromatin is a fundamental aspect of human biology. From regulating gene expression to safeguarding genomic integrity and influencing epigenetic modifications, chromatin structure plays a central role in various cellular processes that impact our well-being.

### SUMMARY

A computer-implemented method is provided. In some embodiments, the computer-implemented method includes: defining an in silico chromatin complex comprising a set of particles, wherein the particles model the in silico chromatin complex as a coarse-grained model; defining an initial epigenetic state based on the structure and/or composition of the chromatin complex; defining a set of force fields, wherein each force field independently corresponds to an interaction between at least one of the particles and at least one of: another one of the particles or a nuclear envelope, or the force field corresponds to an interaction between at least one of the particles and the environment surrounding the in silico chromatin complex; defining a model of an epigenetic modifying process comprising an interaction between the in silico chromatin complex and an external factor, wherein the external factor is capable of changing the initial epigenetic state; accessing molecular dynamics equations comprising the set of force fields; iteratively a) solving the molecular dynamics equations for the in silico chromatin complex and b) applying the model of the epigenetic modifying process; and determining the subsequent epigenetic state resulting from the epigenetic modifying process.

The computer-implemented method may further include structurally characterizing an experimental chromatin complex. The in silico chromatin complex may be defined based on the experimental chromatin complex.

The computer-implemented method may further include using wet lab techniques to measure an experimental epigenetic state of the experimental chromatin complex resulting from the epigenetic modifying process. In some embodiments, the method further includes comparing the experimental epigenetic state to the subsequent epigenetic state. In some embodiments, the wet lab techniques include, for example, measuring histone chemical modifications, measuring histone varieties, measuring chromatin accessibility, or measuring chromatin contact maps, or a combination of the foregoing.

The computer-implemented method may further include evaluating a cost function to obtain a score based on comparing the experimental epigenetic state to the subsequent epigenetic state, and minimizing the score by modifying the set of force fields to define an updated set of force fields.

The computer-implemented method may further include modifying the set of force fields to define an updated set of force fields. The computer-implemented method may further include iteratively a) solving the molecular dynamics equations for the in silico chromatin complex based on the updated set of force fields and b) applying the model of the epigenetic modifying process. The computer-implemented method may further include determining an updated subsequent epigenetic state based on the updated set of force fields. The computer-implemented method may further include comparing the subsequent epigenetic state to the updated subsequent epigenetic state. In some embodiments, the updated subsequent epigenetic state may be more closely correlated to the experimental epigenetic state than the subsequent epigenetic state that was determined prior to modifying the set of force fields.

The computer-implemented method may further include defining a level of granularity for coarse-grained model. In some embodiments, one or more functional groups is modeled as one of the particles or a part thereof in the coarse-grained model. In some embodiments, one or more biomolecules is modeled as one of the particles or a part thereof in the coarse-grained model. In some embodiments, one or more nucleosomes is modeled as one of the particles or a part thereof in the coarse-grained model. In some embodiments, each nucleosome of the in silico chromatin complex is represented by one of the particles.

In some embodiments of the computer-implemented method, the external factor is an epigenetic modifying protein, environment chemistry, or physical forces.

In some embodiments of the computer-implemented method, the initial epigenetic state and the subsequent epigenetic state are each based on the presence or absence of an epigenetic tag at a location of the in silico chromatin complex. In some embodiments, the epigenetic modifying protein is fused to a cas-9 protein. In some embodiments, the epigenetic modifying protein is a methyltransferase.

In some embodiments of the computer-implemented method, the set of simulated time points covers a timespan of about 1 hour to about 6 weeks.

The computer-implemented method may further include modifying the model of the epigenetic modifying process and predicting the effect of the modification on the subsequent epigenetic state.

In some embodiments of the computer-implemented method, determining the resulting epigenetic state comprises applying a probabilistic mathematical model, a deterministic mathematical model, or a set of rules.

In some embodiments, a computer-implemented method is provided. The computer-implemented method can include: defining, for a chromatin molecule, a set of particles in the chromatin molecule; identifying, for each of the set of particles, a location along the chromatin molecule and an estimated epigenetic state; defining a level of granularity for coarse-grained modeling; accessing a set of molecular dynamics equations; and iteratively performing a set of actions that may include: defining a set of force fields using a set of initial force field values or modified force field values, where each of the set of force fields corresponds to an interaction between a particle and at least one of: another particle or a nuclear envelope binding site, where the set of force fields corresponds to a set of particles in an in silico chromatin molecule, and where each of the set of particles may include one or more of the set of particles; generating, for each particle of the set of particles and for each of a set of simulated time points, a predicted position of the particle by performing coarse-grained molecular dynamics simulation using the set of force fields, where the coarse-grained molecular dynamics simulation is performed at the level of granularity and is performed using the set of molecular dynamics equations; generating, using the predicted positions of the set of particles, a set of predicted structural features of the in silico chromatin molecule, where each of the set of predicted structural features identifies a different type of structural feature of the chromatin molecule; accessing, for each predicted structural feature of the set of predicted structural features, a corresponding experimentally measured structural feature measured using a wet-lab experimental technique; generating, for each predicted structural feature of the set of predicted structural features, a score by comparing the predicted structural feature to the experimentally measured structural feature; and determining, for each force field of the set of force fields, a modified force field value using the generated scores and a cost function.

The set of predicted structural features may include a predicted DNA contact frequency. The set of predicted structural features may include a predicted measure of DNA accessibility. Each of the set of force fields may correspond to an interaction between a particle and another particle and a nuclear envelope binding site.

In some embodiments, a computer-implemented method is provided. The computer-implemented method can include defining, for a chromatin molecule, a set of particles in the chromatin molecule; identifying, for each of the set of particles, a location along the chromatin molecule and an estimated epigenetic state; defining a level of granularity for coarse-grained modeling; accessing a set of molecular dynamics equations; defining a set of force fields where each of the set of force fields corresponds to an interaction between a particle and at least one of: another particle or a nuclear envelope binding site, where the set of force fields corresponds to a set of particles in an in silico chromatin molecule, and where each of the set of particles may include one or more of the set of particles in the in silico chromatin molecule; identifying transition criterion that is configured to be satisfied upon detecting a particular characteristic of the in silico molecule; and for each of a set of simulated time points: generating, for each of the set of particles and by using a computational model, a predicted position of the particle; determining whether the transition criterion is satisfied; and upon determining that the transition criterion is satisfied, adjusting an epigenetic state and/or location of a particular particle of the set of particles.

The computer-implemented method may also include: receiving, from a user, input that identifies the transition criterion and identifies the adjustment to the epigenetic state and/or location of the particular particle. The computational model may be a coarse-grained molecular dynamics model. The computational model may include a dynamic model of gene regulatory networks and associated epigenetic modifying proteins.

In some embodiments, a computer-implemented method is provided. The computer-implemented method can include: defining, for a chromatin molecule, a set of particles in the chromatin molecule; identifying, for each of the set of particles, a location along the chromatin molecule and an estimated epigenetic state; defining a level of granularity for coarse-grained modeling; accessing a set of molecular dynamics equations; and iteratively performing a set of actions that may include: defining a set of force fields, where each of the set of force fields corresponds to an interaction between a particle and at least one of: another particle or a nuclear envelope binding site, where the set of force fields corresponds to a set of particles in an in silico chromatin molecule, and where each of the set of particles may include one or more of the set of particles; generating, for each particle of the set of particles and for each of a set of simulated time points, a predicted position of the particle by performing coarse-grained molecular dynamics simulation using the set of force fields, where the coarse-grained molecular dynamics simulation is performed at the level of granularity and is performed using the set of molecular dynamics equations; and for each simulated time point of at least a subset of the set of simulated time points: predicting, using a chemical reaction network and using the predicted positions of the set of particles at the simulated time point, whether a state of an epigenetic tag at a given locus of the in silico chromatin molecule has changed in response to an interaction with an in silico system that may include cas-9 and methyltransferase proteins, where a change in state represents a writing or deleting of the epigenetic tag.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure is described in conjunction with the appended figures:

FIG. 1 illustrates an example of a spring and bead model of a chromatin molecule.

FIG. 2A shows a cartoon representation of a ChIP-seq assay for two histone modifications that may serve as the input for a coarse-grained simulation.

FIG. 2B illustrates an example of an interaction table showing the affinities between all nucleosome combinations and the affinities between all nucleosome and nuclear envelope binding sites.

FIG. 2C is a schematic diagram showing interactions between two nucleosomes harboring specific histone modifications and the nuclear envelope.

FIG. 2D illustrates an example of a simulated chromatin structure that was generated using coarse-grained modeling that included nucleosome and nuclear envelope interactions.

FIG. 3 is a block diagram and example process that updates molecular dynamics force fields by iteratively comparing the output of a molecular dynamics simulation with structural data from a biological chromatin molecule.

FIG. 4A illustrates an example of a ChIP-seq assay for the histone modification H3k4me3 centered around a TERT CpG island along Chromosome 5.

FIG. 4B illustrates an example of the binned data of a ChIP-seq assay for the histone modification H3k4me3 centered around a TERT CpG island along Chromosome 5.

FIG. 4C illustrates an example of the estimated structure of a chromatin molecule generated from the binned data of a ChIP-seq assay for the histone modification H3k4me3 centered around a TERT CpG island along Chromosome 5.

FIG. 4D illustrates an example of a long-range interaction map generated for a chromatin molecule created from the binned data of a ChIP-seq assay for the histone modification H3k4me3 centered around a TERT CpG island along Chromosome 5.

FIG. 4E illustrates an example of an accessibility map generated for a chromatin molecule created from the binned data of a ChIP-seq assay for the histone modification H3k4me3 centered around a TERT CpG island along Chromosome 5.

FIG. 5 illustrates an example of an experiment demonstrating the effects of nuclear envelope stiffness on nucleosome localization within the nucleus.

FIG. 6A illustrates an example of an experiment employing coarse-grained modeling and chemical reaction networks.

FIG. 6B illustrates an example of a simulated genetic engineering experiment where epigenetic modification is restricted to the Chr3q29 locus.

FIG. 7A shows a dot plot result of a control experiment for a simulated genetic engineering experiment. In this simulation, methyltransferase activity was not modeled and there was no epigenetic state conversion.

FIG. 7B shows a dot plot result for a simulated genetic engineering experiment where methyltransferase activity was modeled with a chemical reaction network.

FIG. 7C shows a bar graph revealing the percentage of particles at the periphery for the last time point of a simulated genetic engineering experiment.

### DETAILED DESCRIPTION

### Overview

In some embodiments, the structure of a chromatin complex, also referred to herein as a chromatin molecule, is predicted using coarse-grained modeling and the mathematical modeling technique termed molecular dynamics. While molecular dynamics simulates the movement of particles within a molecule based on the forces that the particles impose on one another, the approach may fail to reproduce the physical properties of large molecules due to their size and complexity. Meanwhile, various disclosures herein improve upon traditional coarse-grained modeling of chromatin by combining an iterative process that compares multiple physical features of the in silico chromatin molecule with those of the biological sample. Estimating the structure of a chromatin molecule may be further improved by considering novel interactions that govern the organization of chromatin, such interactions may include those between nucleosomes and binding sites on the nuclear envelope. Various disclosures herein describe an adaptable system that allows components of the modeled chromatin chain, such as the nature or location of epigenetic modifications, to be altered in real time during the simulation. In some embodiments, the computing system executing the molecular dynamics simulation may respond to external inputs during the simulation and may trigger outputs to interact in real time with a user or other experimental equipment.

### Definitions

As used herein, the term “coarse-grained model” refers to a simplified version of a molecule where functional groups or proteins may be used for molecular dynamics modeling instead of atoms. Herein in, the molecule being simulated is chromatin, which is a complex of DNA and protein found within the nucleus of eukaryotic cells.

As used herein, the term “spring and bead model” refers to a coarse-grained model of chromatin where springs represent DNA molecules and beads represent locations of histones, nucleosomes, or groups of nucleosomes.

As used herein, the term “granularity” refers to the level at which the properties, or epigenetic tags, of adjacent nucleosomes may be combined into a single bead of a spring and bead model. This may, in some instances, also be referred to as binning.

As used herein, the term “particle” refers to a bead in a spring and bead model. Particle, like bead, is a general term that may represent either a single nucleosome, sub-constituents of a single nucleosome, or depending on the level of granularity, a group of adjacent nucleosomes.

As used herein, the term “epigenetic tags” refers to chemical or structural modifications of DNA or DNA binding proteins. Epigenetic tags may, for instance, include histone modifications or molecules that bind to DNA to drive loop formation in the chromatin molecules.

As used herein, the term “molecular dynamics” refers to a mathematical simulation that explores the conformational space of the three-dimensional structure of a molecule. Molecular dynamics simulations calculate the microscopic behavior of a system from the laws of classical mechanics. Molecular dynamics may use, as an input, a description of the interaction potential (or force field) between particles. The typical output of a molecular dynamics simulation may be the location of particles across simulated points in time. Molecular dynamics simulations may reveal the mobility or flexibility of various regions of the molecule or provide the necessary data to quantify how much various regions of the molecule move and what types of structural fluctuations the target molecule undergoes. Molecular dynamics can help infer which chromatin states (i.e., 3D positions) are stable and can be maintained for long periods of time and which states are transitory.

As used herein, the terms “source chromatin chain”, “simulated chromatin chain”, or “biological chromatin chain” refer to biological material that serves as the basis for a coarse-grained simulation. The source chromatin molecule is the biological material on which molecular biology assays such as ChIP-seq, ATAC-seq, and Hi-C may be performed.

As used herein, the term “in silico chromatin chain” or “in silico chromatin complex” refers to the digital or electronic representation of the source chromatin chain that is generated during the course-grained simulation process. Computations that mimic molecular biology assays may be performed on the in silico chromatin chain to compare its structure to that of the source chromatin chain.

As used herein, the term “wet lab” refers to a technique or assay where testing and analyses are performed using physical or biological samples.

### Coarse-Grained Modeling of Chromatin

The three-dimensional organization of DNA, called chromatin, is highly structured and organized within the nucleus of the cell. Strands of DNA wrapped tightly around a family of proteins called histones, which can interact with one another to fold the strands of DNA into complex three-dimensional architectures and structures. Histones assemble into groups of eight to form a structure called a nucleosome, and histone octamers are composed of two copies each of the histone proteins H2A, H2B, H3, and H4. Histone proteins can be modified throughout the life of the cell, and such modifications represent a cell's epigenetic profile. Epigenetic tags serve as a cellular memory for the cell and can be indicative of the cell's biological state and history. As cells mature or undergo diseases (such as cancer) their epigenetic tags are known to change. The compositions of histones and their modifications are important to the health of the cell, as such compositions influence the fine three-dimensional structure of the DNA. Further, the epigenetics of chromatin determines whether transcription factors can bind to the DNA, and thus, the chromatin structure influences gene expression via the regulation of transcription.

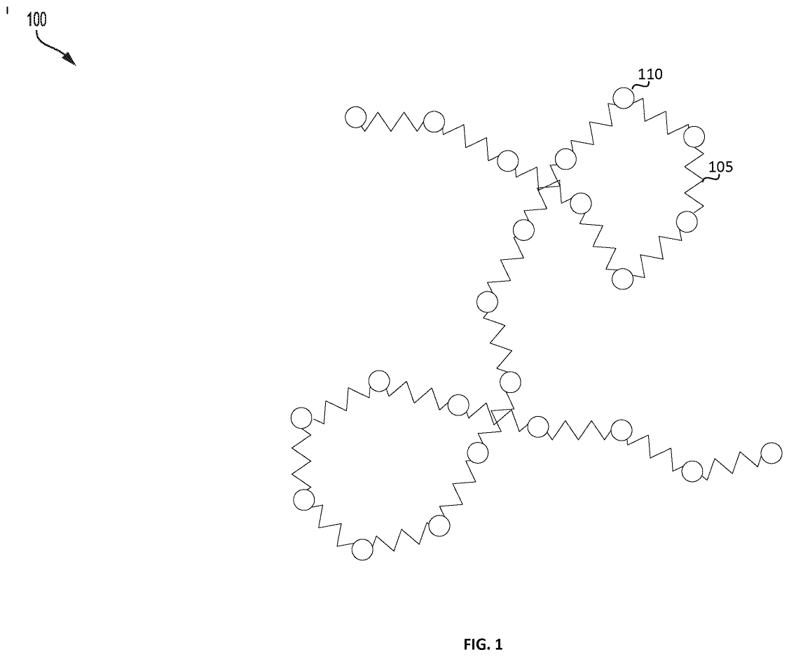

FIG. 1 shows a model of a chromatin molecule where strands of DNA are represented as springs 105 as they may change length (l) and shape by folding, rotating, compressing, and/or twisting in response to applied forces. Factors that contribute to the forces imparted on such DNA strands include changes in histone modifications that may occur locally or even in distal portions of the chromatin. The histone molecules in FIG. 1 are represented as beads 110 connected by sections of open DNA that are not associated with histone molecules. As histone modification is continuously occurring within eukaryotic cells, chromatin is routinely exposed to new forces and thus changing in structure in response.

In some embodiments of the present invention, a coarse-grained model (FIG. 1) is used to represent a three-dimensional structure of chromatin, where the structure accounts for composition of histones within nucleosomes at different points along the DNA. The coarse-grained model may be executed to perform molecular modeling of biomolecules at a given granularity level. The given granularity level may be defined, determined or identified for a given session, input data set, time period, etc. The given granularity level may be determined based on (for example) a selection or identification of a granularity level received via a communication from or interaction via a user device.

The coarse-grained model of chromatin predicts a three-dimensional structure and dynamics of the molecule based on the forces exerted upon the nucleosomes. Features influencing the forces acting on the chromatin include (for example) the location specific nucleosome types along the chromatin chain and the affinity between various nucleosome types within the model. In some instances, interactions between nucleosomes and binding sites on the nuclear envelope are further represented within and used by the model. Stable conformations of the simulated chromatin molecule can be approximated through an iterative process that updates the estimated forces exerted upon the chromatin chain by comparing structural aspects of the coarse-grained model's output with the experimental data from the simulated chromatin chain.

In some embodiments of the invention, the model can reveal changes in chromatin structure in response to modifying an epigenetic tag on a nucleosome during the simulation. The chromatin can be initially modeled with nucleosomes harboring various epigenetic tags. These nucleosomes can be represented along the chromatin chain model in accordance with wet lab experimental techniques, such as ChIP-seq data. Coarse-grained modeling can be used to simulate how the chromatin chain will fold into distinct three-dimensional structures. However, at some time point in the simulation, the identity of an epigenetic tag (the type of nucleosome in the model) may be changed at one or more specific locations in accordance with a modification identified via user input (as further described below). This approach can reveal how specific epigenetic modifications can also influence the movement of nucleosomes within the model, and ultimately the structure of chromatin.

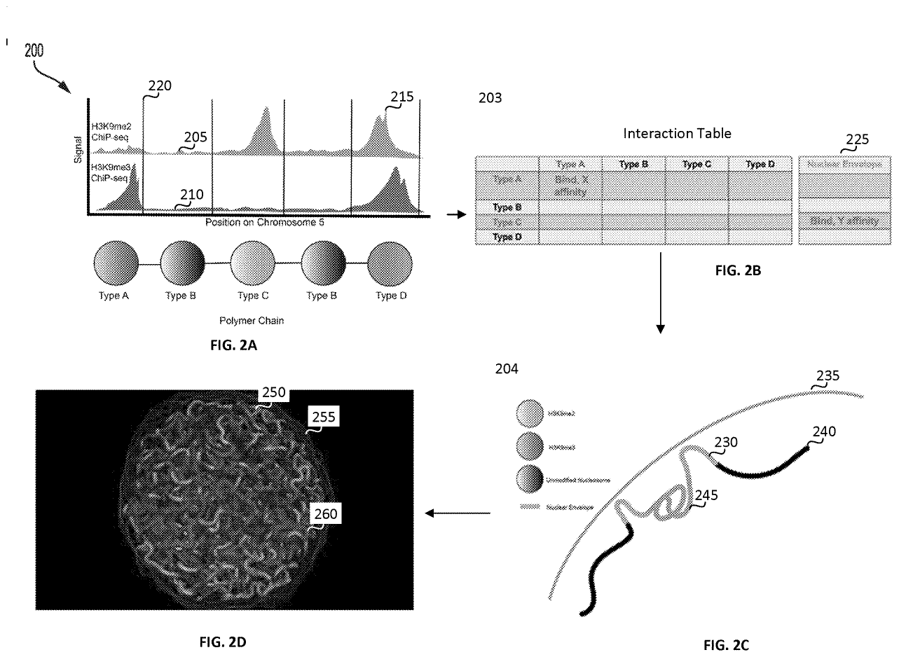

FIGS. 2A-D show exemplary data for creating a coarse-grained model employing the ChIP-seq technique to localize two distinct nucleosome modifications. In FIG. 2A, the orange 205 and blue 210 signals represent the abundance of reads from a ChIP-seq experiment and report the likelihood of a H3K9me2 or H3K9me3 histone modification along a portion of Chromosome 5, respectively. A peak in the signal 215, indicates the likely presence of the histone modification at that location in the source chromatin. In this example, the data is divided into five bins according to the vertical black bars 220. The composition of nucleosomes at the location of the bins is determined by identifying peaks in the signal data and determining which, if any, histone modifications are present in the nucleosome. In this example, a given nucleosome may have the H3K9me3 modification (Type A), neither histone modification (Type B), the H3K9me2 modification (Type C), or both histone modifications (Type D).

The location and identity of epigenetic tags and histone modifications for the coarse-grained modeling may come from assays other than ChIP-seq, including assays such as EpiCypher's Cutana™ Line of Cut&Tag or Cut&Run assays. A result of any technique that provides the locations and compositions of the nucleosomes associated with the chromatin chain may be used as an input to the coarse-grained modeling. Once the location and identity of each nucleosome type is determined, a spring and bead model for the chromatin chain comparable to the example shown in FIG. 1, may be generated. This spring and bead model may be referred to as the in silico chromatin chain.

FIG. 2B shows a sample interaction table that may describe the affinity between types of nucleosomes used in a spring and bead model. The affinity between nucleosome types imparts a force on the chromatic chain and influences how the molecule will fold and move in three-dimensional space. The number of nucleosome types used to generate a coarse-grained model may influence the resolution and fine structure of the final three-dimensional model, but at the cost of the speed of computation. A simulation may include N different states per histone where N is chosen based upon available biological data and the question being investigated. For a chain with K nucleosomes, there are then K^{N} possible states of the chain and N² possible bead-bead interactions.

The nuclear envelope is a double membrane composed of an outer and an inner phospholipid bilayer and is known to possess membrane associated proteins. In various disclosed embodiments of the invention, the interaction table may also include data regarding the affinity between nucleosomes and the nuclear envelope 225. The interactions between nucleosomes and the nuclear envelope are important as they constrain how the chromatin may fold and may influence its three-dimensional structure. The entries in the interaction table can either be determined by (for example) manually curating the interactions from published data or learned using machine learning algorithms that compare model outputs to experimental data, as demonstrated below. The interaction table may include variables that can be translated into rules indicating how different types of nucleosomes are likely to interact; both with each other, and with binding sites on the nuclear envelope.

FIG. 2C shows how specific histone modifications, such as H3K9me2 230, may interact with membrane associated proteins of the nuclear envelope 235, resulting in a structural change in the three-dimensional structure of the chromatin. Unmodified nucleosomes 240 and nucleosomes harboring the H3K9me3 245 modification have a lower affinity for the nuclear envelope and thus do not associate with the membrane. Additionally, nucleosomes bearing the H3K9me3 245 modification exhibit a strong mutual affinity, promoting interactions with each other over interactions with the nuclear envelope.

FIG. 2D shows the structure of a chromatin molecule that was simulated using coarse-grained modeling and the data for the epigenetic modifications depicted in FIGS. 2A-C. Regions of the chromatin that contain nucleosomes with the H3K9me2 modification are shown in green 250 and localize near the thin green circle that represents the nuclear envelope 255 surrounding the chromatin. Regions of chromatin containing unmodified nucleosomes, or those possessing the H3K9me3 modification, are shown in red and do not localize at the nuclear envelope and are more uniformly distributed.

### Molecular Dynamics Simulations

Techniques disclosed herein can generate predictions using a coarse-grained model, which can be theories and equations adapted from molecular dynamics. Molecular dynamic is a computer simulation method for predicting the physical movements of particles across space and time. In coarse-grained modeling of chromatin, atoms and molecules are replaced with histones and nucleosomes. However, the forces between interacting particles may be similarly modeled and used to predict conformational changes to a large molecule. At the core of molecular dynamics is the Langevin Equation:

$$m \ddot{x} = - ∇ U ( x ) - γ m \dot{x} + \sqrt{2 m γ {K}_{b} T} R ( t ) \qquad Equation 1$$

Here, U(x) represents a potential function that includes interactions between particles and location dependent interactions. This function includes the specific force fields that particles experience during the simulation. The function γm\dot{x} is a viscosity term due to interactions with the solvent. Finally, the function \sqrt{2mγK_{b}T}R(t) represents the random force at every time step due to the solvent at temperature T.

The potential function (Equation 2) can be expanded to reveal different potential energies and force fields that will impact the movement of particles, thus governing the chromatin structure. An example of an appropriate potential function of modeling chromatin could take the form of (Equation 2), shown below.

$$U ( x ) = {U}_{bond} + {U}_{lj} + {U}_{angle} + {U}_{bind} + {U}_{confine} + {U}_{nuc} \qquad Equation 2$$

It will be appreciated that, even though Equation 2 shows an exemplary particular approach for calculating U(x), other calculations are contemplated. For instance, it could encompass subsets of these forces and incorporate additional forces for further exploration and comprehension of the fundamental factors governing chromatin dynamics.

Here, U_{bond} (Equation 3) is the bond potential between directly bonded nucleosomes, or particles. This potential can be further represented by the equation:

$$\begin{matrix}1 & \\ {U}_{b o n d} = - \frac{1}{2} {k}_{b} {R}_{0}^{2} ln {( 1 - \frac{{r}_{i j}}{{R}_{0}} )}^{2} & Equation 3\end{matrix}$$

The subscripts i and j are interacting particles. The parameter k_{b} is the force constant or spring constant, typically expressed in units of force per unit. It characterizes the strength of the bond and how stiff or flexible it is. A larger value of k_{b} implies a stronger bond. The equilibrium bond length R₀ is the distance at which the potential energy is minimized. It represents the most stable separation between the two particles in the bond. The actual distance between the two particles involved in the bond is defined as rᵢⱼ and it is the distance for which potential energy will be calculated.

The excluded volume potential, Uₗⱼ, captures the regions of space around particles where other particles are not allowed to enter due to the repulsive interactions between them. The excluded volume potential takes the form of the Lennard-Jones potential energy equation and is shown in Equation 4.

$$\begin{matrix}2 & \\ {U}_{l j} = 4 ε ( {( \frac{σ}{{r}_{i j}} )}^{12} - {( \frac{σ}{{r}_{i j}} )}^{6} ) + ε & Equation 4\end{matrix}$$

In Equation 4, Uₗⱼ is the excluded potential energy at a distance r for two particles, or particles, i and j. The symbol ε is a parameter related to the strength of the repulsion and σ is a parameter related to the size of the excluded volume.

The term U_{angle} from the potential function U(x) represents the potential energy associated with a bond angle deviation and constrains the angle created between three consecutive particles in the model. This potential can be calculated from Equation 5.

$$\begin{matrix}3 & \\ {U}_{angle} = {k}_{a} ( 1 - cos ( θ - {θ}_{0} ) ) & Equation 5\end{matrix}$$

θ represents the actual bond angle between the three particles, and θ₀ is the equilibrium or ideal bond angle at which the potential energy is minimized. The term kₐ represents the force constant or spring constant associated with the bond angle interaction. This force constant determines the strength of the interaction and how stiff or flexible the bond angle is in the coarse-grained model.

The potential function, U(x), may also contain a term, U_{bind}, Equation 6, in this describing the binding potential between particles or epigenetically decorated nucleosomes.

$$\begin{matrix}4 & \\ {U}_{bind} = {α}_{i j} \frac{1}{2} ( 1 + tanh ( μ ( {r}_{shift} - {r}_{i j} ) ) ) & Equation 6\end{matrix}$$

The binding potential between two particles, i and j, is a switch-like activation function. The function is meant to approximate binary binding/unbinding events without having to explicitly create bonds. The term rᵢⱼ is the distance between the two particles. The terms u and r_{shift} shape the binding potential function so that the potential switches quickly from bound to unbound as the particles drift away from each other. This is meant to model protein binding/unbinding (which is short-range, on the order of the size of a protein), or short-range steric repulsion.

The strength or magnitude of the interaction between the two particles is the constant αᵢⱼ. The parameter μ determines the steepness of the transition between bound and unbound. A larger μ value results in a sharper transition, while a smaller μ value results in a more gradual transition. The distance r_{shift} determines where the potential will have half of its maximum magnitude.

As chromatin is naturally confined to the spherical nucleus, a similar constraint can be included in the simulation. This confinement potential restricts the simulation to a spherical volume. The confinement potential is defined as,

$$\begin{matrix}5 & \\ {U}_{confine} = - \frac{1}{3} {{k}_{confine} ( {r}_{confine} - {r}_{i} )}^{3} \times step ( {r}_{i} - {r}_{confine} ) & Equation 7\end{matrix}$$

where k_{confine} is a positive constant that represents the strength of the confinement. It determines how strong the restraining force is on particles when they attempt to move away from the center of the spherical confinement region. A higher value of k_{confine} results in a stronger restraining force. The parameter r_{confine} is the radius of the spherical confinement region, or this case, the radius of the nucleus. It specifies the distance from the center of the sphere to its boundary. Particles, or nucleosomes, are constrained to stay within this radius. Any particle attempting to move beyond this distance will experience a force pulling it back toward the center of the sphere.

The distance of the particle i from the center of the spherical confinement region is the term rᵢ. It is a dynamic variable that changes as the particle moves within the simulation. The step function, step(rᵢ−r_{confine}) returns 1 if rᵢ is greater than r_{confine}, and it returns 0 if rᵢ is less than or equal to r_{confine}. In other words, it is a function that switches on when the particle, or nucleosome, is outside the spherical confinement, or nuclear envelope, and remains off when the particle is inside. The term (r_{confine}−rᵢ)³ represents the restraining force applied to a particle when it attempts to move beyond the confinement boundary. When a particle is outside the confinement (rᵢ>r_{confine}), this term becomes positive and increases as the particle moves further away from the center. It effectively acts as a restoring force that pulls the particle back toward the center of the spherical confinement region.

The spherical confinement function thus applies a restraining force, proportional to k_{confine}, to particles when they are outside the specified spherical region when (rᵢ>r_{confined}). This force becomes stronger as particles move further from the center of the nucleus. The purpose of this function is to ensure that chromatin remains confined within the specified spherical region during the simulation.

Some embodiments of the invention relate to an improved molecular dynamic simulation that accounts for nuclear potential. For example, Equation 8 shows an exemplary formula for calculating a nuclear potential:

$$\begin{matrix}6 & \\ {U}_{n u c} = {α}_{i - n u c} \frac{1}{2} ( 1 + tanh ( {μ}_{n u c} ( {r}_{shift} - {r}_{i - n u c} ) ) ) & Equation 8\end{matrix}$$

The nuclear potential function takes a similar form as the binding function described above, but instead describes the interaction between a particle and a nuclear envelope membrane protein rather than between two particles. The strength of the interaction between a nucleosome, or particle, and a binding site on the nuclear envelope is thus the constant α_{i-nuc}. Likewise, the parameter μ_{nuc} determines the steepness of the transition between bound and unbound interactions. The distance between a nucleosome and a binding site on the nuclear envelope is defined as r_{i-nuc} and the distance r_{shift} represents the distance where the potential will have half of its maximum magnitude.

Though specific equations are provided herein as examples for estimating potential variables, other equations may be contemplated. For example, Equation 2 can be modified to calculate the potential U(x) using one or more additional potentials not represented in Equation 2 and/or in a manner that does not depend on at least one potential shown in Equation 2. In some embodiments, the potential U(x) is defined to be a sum of multiple potentials, where the multiple potentials include a nuclear potential U_{nuc}. The inclusion of U_{nuc} may be important in that nucleosomes are known to interact with membrane-bound proteins associated with the nuclear envelope. As FIGS. 2A-D illustrate, interactions between nucleosomes and the nuclear envelope influence the three-dimensional structure of the chromatin molecule.

In some embodiments, the nuclear envelope is explicitly modeled and is also free to undergo motion or changes in the nature of its bead types. The bead types in this case could represent the presence of specific nuclear envelope proteins for example.

In summary, molecular dynamics can be used to simulate the movement of particles, or in this case nucleosomes, in a coarse-grained model, and to provide insight into movement trajectories and equilibrium points. The trajectories of particles in the simulation are determined by the forces acting upon them, and these forces are largely governed by the value of the parameters and constants within the potential function. The selection of values for parameters and constants within the potential function can be selected so that molecular dynamics trajectories closely resemble (e.g., most closely resemble) the trajectories observed in biological data.

The output of the molecular dynamics simulation may include the position of particles, or nucleosomes, in the simulation at points in time. The simulation may track how often particles contact each other, or a nuclear envelope binding site, to build a contact probability matrix. The contact probability matrix is comparable to experimental Hi-C data. Data from the simulation can also be used to calculate the local density along the chromatin molecule and to produce accessibility data (accessibility is the inverse of density) comparable to experimental ATAC-seq.

### Iterative Data-Driven Simulation for High Accuracy Three-Dimensional Modeling

While proving to be a powerful technique, coarse-grained modeling is unfortunately susceptible to sources of errors that impact the dynamics of the particles being simulated. Common sources of errors in coarse-grained modeling are described below. These include but are not limited to:

Choice of Coarse Graining Level: Coarse graining involves simplifying a system by representing groups of atoms or molecules as single entities. This may include the resolution of bulk histone modification date. If the level of coarse graining is too high, important details may be lost.

Force Field Parameters: Coarse-grained models rely on force fields, which include parameters (such as interaction strengths) that are often fitted to reproduce specific properties of interest. The parameters are captured in the potential equation described above. Errors can arise if the force field parameters are not accurately determined or do not adequately represent the system being studied.

Choice of Potential Functions: The choice of potential functions used to model interactions between coarse-grained particles can impact accuracy. If the potential functions are not chosen appropriately or are not capable of accurately describing the interactions in the system, errors can occur.

Boundary Conditions: The choice of boundary conditions in simulations can introduce errors. If the system size is too small, finite size effects can lead to errors. Likewise, if the boundary conditions do not accurately reflect the physical system, errors can arise.

Time Step and Integration Scheme: Errors can arise from the choice of the time step used in molecular dynamics simulations and the numerical integration scheme employed. If the time step is too large or the integration scheme is not sufficiently accurate, the simulation may not capture the dynamics of the system correctly.

Thermodynamic Ensemble: Depending on the ensembles used to control the thermodynamic variables during the simulation (e.g., NVE Ensemble [number of particles, volume, energy constant], NVT Ensemble [number of particles, volume, temperature constant], or NPT Ensemble [number of particles, pressure, temperature constant]), different errors can arise. For example, maintaining temperature and pressure can introduce errors if not done correctly.

Initial Conditions: The choice of initial conditions for simulations can have a significant impact on results. If the initial configuration or velocities are not representative of the system's actual behavior, it can lead to incorrect outcomes.

Statistical Sampling: Coarse-grained simulations are often based on statistical mechanics principles, and errors can arise from inadequate sampling. Running simulations for insufficiently long times or not sampling the configuration space adequately can lead to errors in calculated properties.

Given the number of error sources, modeling the structure of chromatin using a coarse-grained modeling approach clearly represents a technical challenge in need of a technical solution. In some embodiments, the invention includes an iterative method to validate the output of coarse-grained modeling of chromatin, in which the computer-implemented method can be deployed as software on a computing device. The computer-implemented method includes at least two forms of experimental validation, such as data examining long-range nucleosome interactions and data relating to DNA accessibility or similar measurements. Long-range chromatin interactions can be derived experimentally using various assays including but not limited to 3C (Chromosome Conformation Capture), 4C (Circular Chromosome Conformation Capture), or Hi-C(High-throughput Chromosome Conformation Capture). DNA accessibility profiles can be generated using multiple approaches and strategies, such as ATAC-seq, DNase-seq, MNase-seq, FAIRE-seq, etc. Alternative approaches may include techniques, such as Kas-seq, which measures the single-stranded DNA produced at regions of active transcription, thus providing a measurement of activity complementary to DNA accessibility.

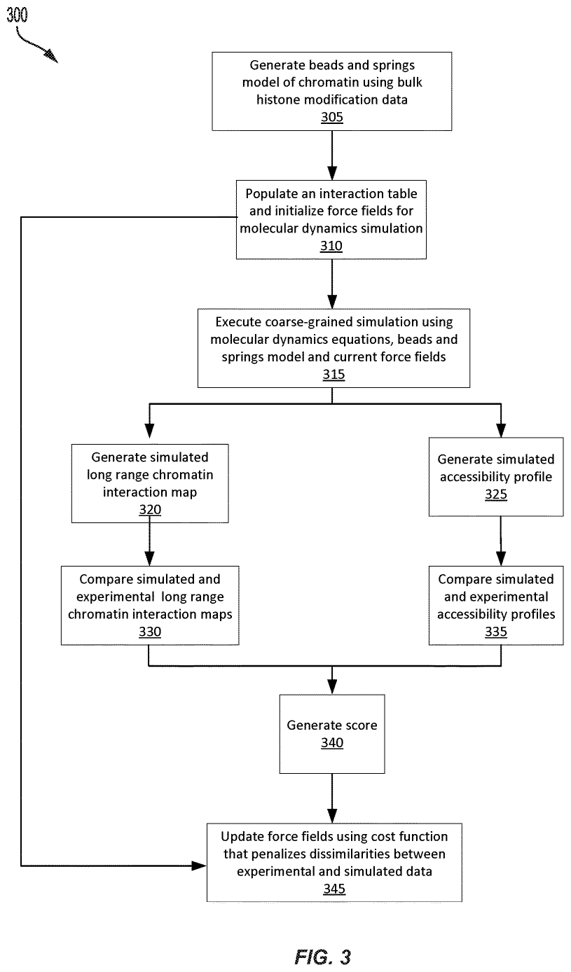

FIG. 3 illustrates an exemplary iterative process 300 that estimates the movements and positions of nucleosomes in a coarse-grained model of chromatin. More specifically, force field values that are used for molecular dynamics simulations are updated by comparing the output of the simulation with experimental data from the source chromatin being modeled.

At block 305, a beads and springs model of a chromatin molecule is generated using bulk histone modification data. The bulk histone modification data includes the location and epigenetic state of nucleosomes and may be determined using experimental approaches, such as ChIP-seq. The beads and springs model can include a set of particles, with each particle representing one or more nucleosomes and a state (e.g., a default state or a modified state representing a particular epigenetic state). The beads and springs model can be configured in accordance with a specific granularity level that indicates how many adjacent nucleosomes are to be represented by a single particle in the in silico chromatin chain. The granularity level may be defined based on received user input.

At block 310, an interaction table is populated, and force fields are initialized for a molecular dynamics simulation. The interaction table can include a set of predicted affinities between a particle and either another particle or the nuclear envelope. An exemplary interaction table is shown in FIG. 2B.

At block 315, a coarse-grained simulation is executed using the current force fields and a set of molecular dynamics equations. The molecular dynamics equations may include a potential function equation defining one or more forces identified herein. The potential function may include the equation for the nuclear potential describing the interaction of epigenetically decorated nucleosomes (or particles representing binned nucleosome data) and binding sites on the nuclear envelope. The coarse-grained simulation may calculate a position of each particle across increments in time until a set amount of simulated time has passed or until a criterion (e.g., defined by input from a user device) has been satisfied. After block 315, the process 300 continues to block 320 and/or 325. Blocks 320 and 325 may be executed in any order and in series or parallel.

At block 320, the process 300 generates simulated long-range interaction maps for the in silico chromatin chain. Long-range interaction maps may be generated by mimicking techniques including but not limited to 3C, 4C, Hi-C, ChIA-PET (Chromatin Interaction Analysis by Paired-End Tag Sequencing), etc. For example, to generate a Hi-C map from the in silico chromatin chain, block 320 may include extracting information about the spatial interactions between different regions of the chromatin chain. This information may include the distances or contacts between chromatin domains or particles. The chromatin chain may be divided or binned into discrete genomic bins or segments. The size of these bins may correspond to a resolution of experimental Hi-C data. Typically, experimental Hi-C data is binned at various resolutions, such as 1 kb, 5 kb, or 10 kb. For each pair of genomic bins, the number of interactions or contacts observed in the coarser-grain simulation is counted. This may involve calculating the number of times that pairs of particles in the simulation come into close spatial proximity. Interaction counts may be normalized to correct for biases and experimental noise. Normalization methods used in real Hi-C data, such as iterative correction, can be adapted for simulated interactions. With the normalized interaction counts, the process 300 can generate a Hi-C-like contact matrix. This matrix represents the frequency of interactions between genomic bin pairs.

At block 325, a simulated accessibility profile for the in silico chromatin chain may be generated. Accessibility with regards to a chromatin chain can be modeled as the inverse of the local density along the chain. The local density can be estimated based on the predicted position of the particles. Thus, at block 325, the local density along the in silico chain can be calculated as the number of particles within a specified volume. The inverse of the local density may then be stored as the accessibility at that position along the chromatin chain.

At block 330, the long-range interaction maps generated from the in silico chromatin chain may be compared to the source chromatin chain. A similarity metric for long range association data, such as Hi-C data, may be computed using a variety of techniques and/or algorithms. For example, a differential interaction analysis tool (diffHiC, HiCcompare, HiCRep, etc.), differential compartment analysis tool, visualization tool, principal components analysis, correlation analysis, permutation testing, normalization, significance thresholding, or machine learning tool (e.g., random forest or support vector machine) can be used to classify or predict differences between Hi-C maps based on various features or patterns. The comparison may result in generating an interaction-comparison metric, which can be a numeric (e.g., real-number of integer) metric.

At block 335, the accessibility profile generated from the in silico chromatin chain is compared to an experimentally measured accessibility profile of the source chromatin chain. The experimentally measured accessibility profile can be generated using a wet lab technique such as ATAC-seq. Comparisons may be made using normalization methods, differential accessibility analysis (DESeq2, edgeR, DiffBind, csaw, etc), or functional enrichment analysis (using tools such as DAVID, Enrichr, clusterProfiler, etc), among other options. The comparison may result in generating an accessibility-comparison metric, which can be a numeric (e.g., real-number of integer) metric.

At block 340, a score is generated based on the comparisons performed at blocks 330 and 335. Thus, the score may be based on comparisons of multiple metrics corresponding to different predicted structural features. Though blocks 320-335 relate to metrics corresponding to two particular structural features, it will be understood that one or more comparisons may alternatively or additionally be performed based on another structural feature. The score may be generated by (for example) by calculating a statistic based on the metrics generated during the comparisons performed at blocks 330 and 335 (or based on one or more other comparisons). The statistic may include (for example) a weighted or unweighted average or median.

At block 345, the force fields can be updated using the generated score and a cost function. The cost function can be configured to generate a penalty that correlates with dissimilarities between the structure of the source chromatin chain and the in silico chromatin chain (based on the combined metric scores). The force fields for the potential function can be updated in a manner that minimizes the penalty. Upon completion of block 345, the process 300 executes block 310 and continues to loop between blocks 310 and 345 until a given number of cycles (e.g., a predefined number or a number as identified via input received from a user device) are completed or until another condition is satisfied (e.g., where a score exceeds a given threshold).

The process 300 is thus iterative and functions to minimize discrepancies between in silico and experimental wet lab data by updating the affinity values in the interaction table, and thus the force fields in the potential function. The process 300 reduces or minimizes errors in modeling chromatin's three-dimensional structure in at least three different manners. First, as described above, the process 300 is iterative and updates force field values for the interactions table to minimize discrepancies between simulated data and experimental data. Second, the process 300 utilizes multiple physical features of chromatin structure to evaluate the performance of the simulation and to minimize discrepancies between simulated and experimental data. Such data may include but is not limited to data regarding long-range DNA interactions and accessibility profiles. Third, the process 300 incorporates molecular interactions between nucleosomes and the nuclear envelope proteins. By combing these strategies, various techniques disclosed herein increase the accuracy of modeling the three-dimensional structure of chromatin in a coarse-grained model.

As various techniques disclosed herein (e.g., relating to part or all of process 300) can estimate high resolution three-dimensional structures of chromatin, it may be employed to analyze the association between epigenetic tags and development, aging, or diseases where epigenetic modifications are common, such as cancer. As the structure of chromatin is a major factor in regulating gene expression, the high-resolution models are also valuable for research in genetics and gene regulation.

A feature of the process 300 that extends its applicability is the inclusion of nucleosome and nuclear envelope protein interactions, as indicated in the interaction table show in FIG. 2B. This feature serves at least two purposes. As described above, it greatly improves the accuracy of the three-dimensional reconstructions of the chromatin. This highlights the importance of such interactions in governing the structure of chromatin. Second, it may provide insights as to how such interactions alter chromatin structure, and thus gene expression. Histones with unique epigenetic modifications have distinct affinities for nuclear envelope proteins. Thus, altering epigenetic tags can affect gene expression by altering chromatin structure via nucleosome/nucleosome interactions or nucleosome/nuclear envelope protein interactions. Alternatively, if an experimentalist or clinician aims to alter chromatin structure as a method for regulating gene expression, the process may reveal how changes to the composition of the nuclear envelope might alter the structure of chromatin.

Some implementations of the process 300 may thus be viewed as nested loops. The inner-most loop (at block 315) represents the molecular dynamics simulation and the outer loop corresponds to actions for comparing the simulation data to experimental data and updating force-field values to reduce errors in the calculated three-dimensional structure of the simulated chromatin molecule. Thus, the process can include a set of actions that: 1) configures and executes a molecular-dynamics simulation, 2) compares the model output to experimental data 3) updates the interaction table as needed, and 4) repeats, until a condition is satisfied (e.g., a suitable level of convergence is achieved between the simulated and experimental data, a threshold number of iterations have been performed, or a desired amount of time has passed). Upon completion of the process 300, data from the coarse-grain simulation 315 that was generated from one or more iterations of the process 300 may be used to provide information regarding the structure and conformational space of the simulated chromatin molecule. As described above, this data may include, for each particle, its position and/or state (e.g., epigenetic state) at each of one or more points in time (e.g., at a final point in the simulation). The interim, final and/or optimized force field values can be stored in one or more versions of the interaction table FIG. 2B. Access to the final interaction table values allows the user to now perform the coarse-grained simulation on the data set without the need to perform the entire optimization loop. This can be useful (for example) to inform subsequent user inputs that trigger changes to the simulated chromatin molecule (for instance, the location of an epigenetic tag along the chain, etc.).

### Editable Molecular Dynamics for Coarse-Grained Modeling

Some actions of the present invention may occur outside the depicted loop between blocks 310 and 345 of process 300 and instead may occur during a subsequent simulation or processing that utilizes the finalized force fields. Such action(s) may include editing properties of the simulated chromatin chain, such as the identity of an epigenetic tag, during the simulation or to interact with the input/output (I/O) component of a computing system executing the simulation.

Updates to the coarse-grained simulation may occur at any relevant point in time. The relevant point in simulation time for interruption may be predetermined prior to initializing a first cycle of a coarse-grained simulation or triggered by any predefined condition. For instance, an interface can be configured to receive a user input (e.g., in real time or in advance) that indicates how (e.g., and/or when) the identity of an epigenetic tag at a specific location of the in silico chromatin chain is to change at a given time step of the simulation.

There are several manners in which in which epigenetic tags may vary in the simulation. As a first example, a predefined perturbation may be defined via computer code which automatically changes the epigenetic tags at a particular position subject to any condition that can be written as computer code. A more biologically motivated version of the previous example is to use a dynamical model of gene regulatory networks and associated epigenetic modifying proteins to automatically change the epigenetic tags. For instance, the amount of a histone-acetylate may be simulated within the cell and dynamically acetylate histones in the chromatin model, changing the epigenetic tags. Note that both of these kinds of dynamical changes to epigenetic tags in the model occur in real time within the simulation and do not require human intervention (although code could be written such that humans are able to actively interrogate/modulate epigenetic tags and visualize the results).

The ability to alter epigenetic states during molecular dynamics experiments can aid provide information as to why some cells may fail to reprogram upon modification of their epigenome. For example, available data may include Chip-Seq data measuring epigenetic marks at two time points during a process of interest (e.g., Day 1 of a reprogramming experiment and Day 7 of a reprogramming experiment, etc.). Data from Day 1 can be used to generate the initial chromatin configuration, and subsequently simulate the transition to the epigenetic state associated with the data on Day 7. Determining why some cells fail to reprogram could be done by comparing the chromatin trajectories of successfully reprogrammed cells to cells that fail to reprogram. Learning when and why chromatin structural transitions fail may inform the optimization of reprogramming experiments.

Some embodiments of the invention may also include the ability to trigger an output from the computing system that is performing the molecular dynamics simulation. This output may take on many forms and serve varied purposes. In some instances, this may simply be a notification transmitted to or availed at a user device that indicates that the simulation has reached a certain time point in the simulation or that the in silico chromatin chain has achieved a specific conformation. The output, if configured during the process 300, may also serve to let the user know that the force field estimates are changing minimally, suggesting that the process 300 is nearing completion. This may indicate that the simulations are converging on force field values that minimize the penalty of the loss function from block 345. Finally, the output signal from the computing system may take the form of a digital signal that may be used to trigger an external process. For instance, at a certain point in the simulation, the computing system can output a voltage pulse that can be interpreted by another piece of experimental equipment to perform a function.

In some embodiments a trigger generated from the chromatin simulation may be used to couple the dynamics of chromatin to other dynamical in silico models. For instance, in a whole-cell model, certain epigenetic triggers may be required before a separate model of cell division is activated. In such an experiment, the two or more in silico models may be executed on the same computing system or different computing systems.

Techniques disclosed herein can provide improved coarse-grained models of chromatin that can guide sophisticated computer-aided epigenetic engineering. Combining such simulations with these experimental epigenetic engineering tools could be fundamental in developing therapies that modulate the epigenome to reverse disease. An additional contribution of this present disclosure lies in the shift away from the binary perspective on chromatin as a molecule with only two states, heterochromatin and euchromatin. Instead, it recognizes a multitude of states, contingent upon their biological relevance.

### Chemical Reaction Networks and Targeted Epigenetic Engineering

In certain embodiments of the invention, molecular dynamics simulations can be integrated with a chemical reaction network that details additional molecular biology events potentially affecting the nature of epigenetic tags or chromatin configurations. A chemical reaction network provides a mathematical representation of the interactions and transformations among various chemical entities, highlighting the network of chemical pathways and reactions inherent to a designated system.

Integrating chemical reaction networks with coarse-grained chromatin modeling may be useful, especially when planning precision epigenetic engineering experiments. Such experiments may aim to modify the epitope tag of a nucleosome at a distinct chromatin chain location. Given that chemical reactions, including biological ones, inherently possess probabilistic characteristics, simulations that address the stochastic essence of both the biochemical interactions and the physical chromatin configurations enhance the statistical robustness of outcome predictions.

An example of such an epigenetic engineering experiment may utilize the enzymes dCas9 and the G9a methyltransferase to alter an epigenetic tag, where the dCas9 component provides site specificity and the methyltransferase component alters the epitope tag chemically. The protein dCas9 is an RNA-guided endonuclease enzyme component of the bacterial defense mechanism against viruses and may be adapted for genetic engineering experiments. The guide RNA in this system may be designed to target specific regions of DNA near the known location of a histone that is targeted for epigenetic modification. The dCas9 molecule may be engineered in two aspects. First, the enzymatic properties of the dCas9 protein may be rendered non-functional to preserve the integrity of the targeted region of DNA. Second, the dCas9 molecule may be made into a fusion protein harboring the G9a methyltransferase domain. The G9a methyltransferase domain can write the H3K9me1/2 epitope tag onto nearby histones. Thus, in this system, the guide RNA and dCas9 molecules provide the location specificity, and the methyltransferase component provides the chemical ability to rewrite epigenetic tags on nearby histones. Both elements of the system—the formation of the dCas9, guide RNA, and DNA complex, and the methyltransferase enzyme activity—are probabilistic chemical reactions, making them suitable for chemical reaction network modeling.

In some embodiments, a chemical reaction network is used to represent the G9a methyltransferase activity on a specific nucleosome or particle during the coarse-grained simulation of a chromatin chain using a spring and bead model. The complexity of this network can differ based on which components are included. For instance, some models might only include the final methyltransferase component, while others might also include the dCas9 complex. In certain models, the methyltransferase activity can be reversible, allowing epigenetic changes to revert at a set rate. However, in simpler models, once an epigenetic change is made, it persists for the duration of the simulation.

Simulations combining coarse-grained modeling of chromatin and chemical reaction networks mimicking dCas9 methyltransferase activity may serve multiple purposes. First, they may help estimate the efficiency of epigenetic tag modification at different sites along the chromatin as a function of the three-dimensional chromatin state. Some sites may be more easily modified whereas others much be occluded. Alternatively, such simulations may demonstrate how successful epigenetic conversion can instead alter the three-dimensional structure of chromatin, potentially by relocating certain particles, or nucleosomes, to the nuclear envelope.

### EXAMPLES

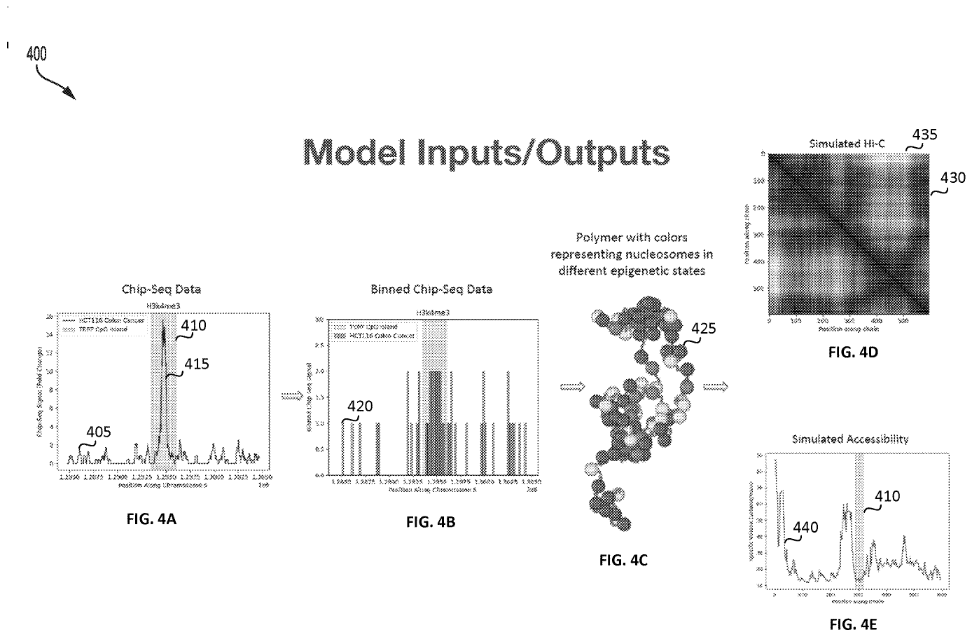

FIGS. 4A-E illustrates exemplary data transformations 400 that generate multiple predicted structural data sets. Bulk histone modification data from a ChIP-seq experiment 400 is shown for the H3K4me3 epigenetic modification in the HCT116 human colon cancer cell line. FIG. 4A shows the intensity signal 405, or read count, for the H3K4me3 epigenetic tag. The plotted data is restricted to a portion along Chromosome 5 that includes the TERT CpG island 410 shown as a gray box.

TERT CpG refers to a specific region in the human genome that contains cytosine (C) and guanine (G) base pairs (a CpG dinucleotide) within the promoter region of the TERT gene. The TERT gene encodes the telomerase reverse transcriptase enzyme, which is a critical component of the telomerase complex responsible for maintaining and elongating telomeres. In the acquisition of colon cancer, the structure of chromatin changes as histones in the nucleosomes at the TERT CpG island acquire the H3K4me3 epigenetic tag. This modification is believed to be associated with changes in gene expression. In colon cancer cells, elevated H3K4me3 levels at the TERT promoter can enhance the expression of the TERT gene. Increased TERT expression can be a crucial factor in cancer because it allows for the continued maintenance and elongation of telomeres. This, in turn, helps cancer cells evade senescence and continue to divide, contributing to the cancer's growth and progression. The data in experiment 400 shows a peak 415 in the TERT CpG island 410, demonstrating that this region is enriched in H3K4me3 modified histones compared to neighboring regions.

ChIP-seq data can be used directly for the coarse-grained modeling to obtain single nucleosome resolution (or approximately 200 base pair resolution) as demonstrated in FIG. 4A. Alternatively, the nucleosome data may be binned across neighboring nucleosomes to increase the speed of simulation, as was done in the example in FIG. 4B. In this experiment 400, the binned data 420, location and epigenetic states of nucleosomes, was then imported into the simulation along with an appropriate interaction table FIG. 2B. The epigenetic states of the particles 425, or binned nucleosomes, are shown in different shades of gray in the beads and strings model of chromatin in FIG. 4C.

Data from the simulation was used to generate two data sets. FIG. 4D shows simulated Hi-C data for comparison with experimental data. Darker signals 430 show a strong interaction between particles along those of the chromosome. Lighter regions 435 of the simulated Hi-C map are indicative of little direct interaction between those regions of the chromosome.

The process and the output of the simulation also returned the simulated accessibility data shown as the black trace 440 in FIG. 4E. A drop in accessibility, consistent with an increase in chromatin density, is shown at the TERT CpG island 410. The simulated accessibility data can be directly compared to experimentally derived ATAC-seq data for the same chromosome region.

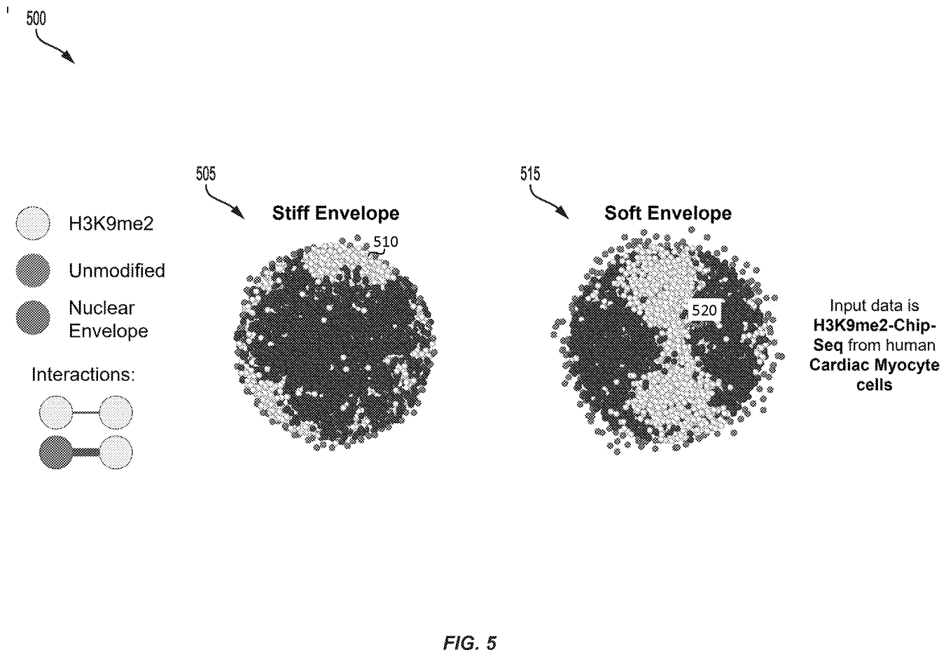

Example 2 shown in FIG. 5 illustrates how altering the composition of the nuclear envelope can transform the three-dimensional structure of chromatin. In this experiment, the process 300 was used to model the chromatin structure of H3K9me2 ChIP-seq data from human cardiac myocytes. The position of nucleosomes with unmodified histones are shown as blue circles and modified nucleosomes bearing the H3K9me2 tag are shown in white. The nuclear envelope is represented as red circles and is modeled as a mesh-like network of beads connected by springs. The interaction between H3K9me2 harboring nucleosomes is weaker than the interaction between an H3K9me2 harboring nucleosome and a nuclear envelope binding site. In the control condition 505, nucleosomes with the H3K9me2 modification tend to associate near the nuclear envelop 510. However, softening the nuclear envelope 515 by reducing the spring constant of the bonds that connect the nuclear envelope beads together alters the interaction between nuclear envelope beads and H3K9me2 modified nucleosomes. Softening the envelope causes H3K9me2 modified nucleosomes to move away from the envelope towards the center of the nucleus 520. Since the nuclear envelope bead motion is also being simulated, this softening leads to larger oscillations of the nuclear envelope, which disrupts the ability for chromatin to bind to the envelope beads. This experiment demonstrates how the method may be used to not only model the three-dimensional structure of chromatin, but also how the method may help predict how perturbations of the nuclear envelop can be used to regulate gene expression via altering chromatin in a predictable manner.

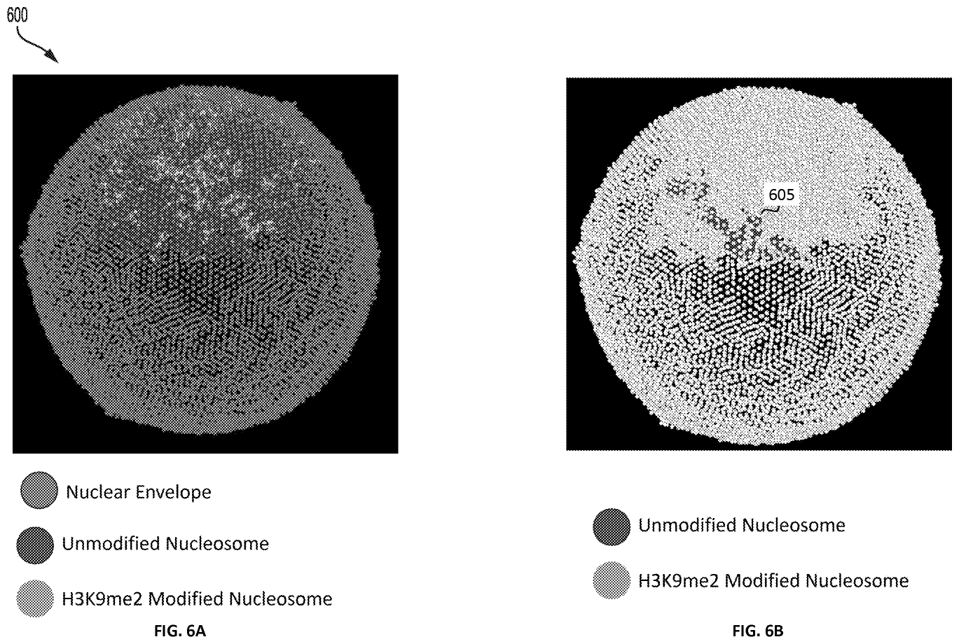

Experiment 3 presents results from an integrated simulation of epigenetic engineering, combining coarse-grained modeling with a chemical reaction network 600. This visualization provides both the position and state of each particle at the start of the simulation. In FIG. 6A, the red beads depict binding sites on the nuclear envelope, modeled as a fluid mesh-like network, while blue beads symbolize unmodified particles or nucleosomes of Chromosome 3 using the bead and spring model. Cyan beads represent H3K9me2 modified nucleosomes.

In FIG. 6B, the color of the nuclear envelope binding sites has been changed to white for improved clarity. Here, the blue beads specifically indicate nucleosomes associated with the Chr3q29 locus of Chromosome 3. The simulation's chemical reaction network sets the likelihood of these specific nucleosome sites converting to the H3K9me2 epigenetic state. This targeted simulation approach echoes the dCas9 experimental method used in real-world genetic engineering, where the chemical reaction network mirrors the likelihood of nucleosome tag changes due to methyltransferase activity in biological experiments. In this context, the blue beads in FIG. 6B are the subset of particles, or nucleosomes, in the simulation that are available for modifications. Conversely, the cyan beads represent particles that began the simulation in the H3K9me2 state, which is an emulation of the natural H3K9me2 modification rate at the Chr3q29 locus.

Upon running the simulation, the conversion rate of nucleosomes at the Chr3q29 locus to the H3K9me2 state is determined by the parameters set by the chemical reaction network. Overall, such simulations may sheds reveal on how specific epigenetic modifications may affect the structure and dynamics of chromatin molecules.

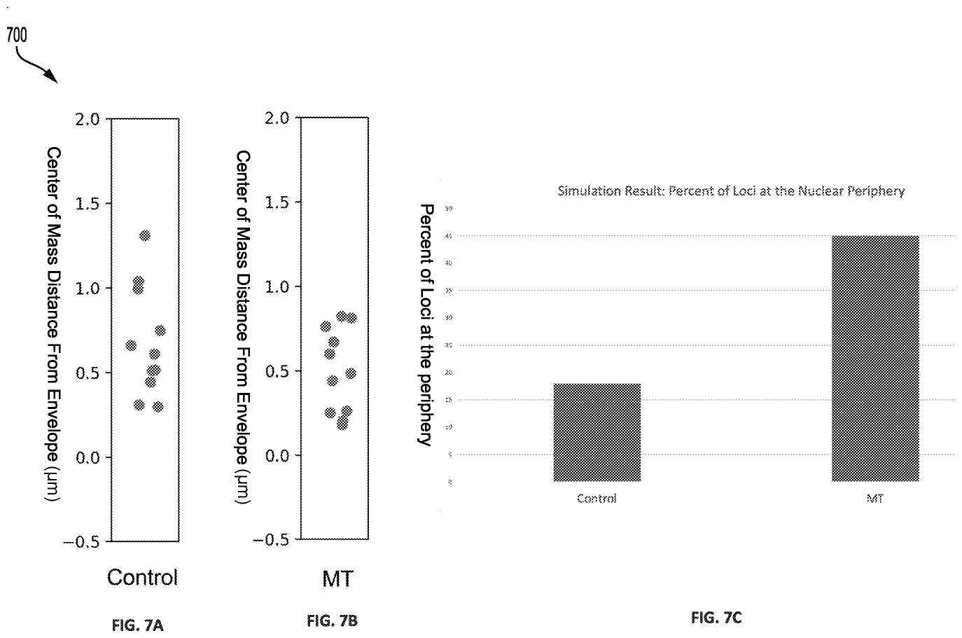

FIGS. 7A-C show the results of the experiment 600. As a control experiment (Control), the simulation was configured to not include methyltransferase activity, or conversion to the H3K9me2 epitope tag. The distance of particles in the Chr3q29 locus from the nuclear envelope in the control experiments are shown in FIG. 7A. The data in FIG. 7B shows a similar simulation, but where methyltransferase activity as simulated within the Chr3q29 locus of the chromatin chain (termed MT). The rate of conversion to the H3K9me2 epitope tag phenotype was controlled by a chemical reaction network. Comparing the data in FIGS. 7A and B reveals that conversion of particles to the H3K9me2 modified state decreases their distance to the nuclear envelope. The bar graph shown in FIG. 7C further demonstrates this observation by showing the percentage of particles at the Chr3q29 locus which localize to the periphery, or nuclear envelope, in the Control and MT simulations. When methyltransferase activity is included in the simulation, a greater percentage of particles at the Chr3q29 locus localize to the periphery.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the present description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

It is claimed:

1. A computer-implemented method comprising:
defining an in silico chromatin complex comprising a set of particles, wherein the particles model the in silico chromatin complex as a coarse-grained model;
defining an initial epigenetic state based on the structure and/or composition of the chromatin complex;
defining a set of force fields, wherein each force field independently corresponds to an interaction between at least one of the particles and at least one of: another one of the particles or a nuclear envelope, or the force field corresponds to an interaction between at least one of the particles and the environment surrounding the in silico chromatin complex;
defining a model of an epigenetic modifying process comprising an interaction between the in silico chromatin complex and an external factor, wherein the external factor is capable of changing the initial epigenetic state;
accessing molecular dynamics equations comprising the set of force fields;
iteratively a) solving the molecular dynamics equations for the in silico chromatin complex and b) applying the model of the epigenetic modifying process; and
determining the subsequent epigenetic state resulting from the epigenetic modifying process.

2. The computer-implemented method of claim 1, further comprising structurally characterizing an experimental chromatin complex, wherein the in silico chromatin complex is defined based on the experimental chromatin complex.

3. The computer-implemented method of claim 2, further comprising using wet lab techniques to measure an experimental epigenetic state of the experimental chromatin complex resulting from the epigenetic modifying process; and
comparing the experimental epigenetic state to the subsequent epigenetic state.

4. The computer-implemented method of claim 3, wherein the wet lab techniques comprise measuring histone chemical modifications, measuring histone varieties, measuring chromatin accessibility, or measuring chromatin contact maps, or a combination of the foregoing.

5. The computer-implemented method of claim 1, further comprising evaluating a cost function to obtain a score based on comparing the experimental epigenetic state to the subsequent epigenetic state, and minimizing the score by modifying the set of force fields to define an updated set of force fields.

6. The computer-implemented method of any one of claim 1, further comprising modifying the set of force fields to define an updated set of force fields.

7. The computer-implemented method of claim 1, further comprising iteratively a) solving the molecular dynamics equations for the in silico chromatin complex based on the updated set of force fields and b) applying the model of the epigenetic modifying process; and determining an updated subsequent epigenetic state based on the updated set of force fields.

8. The computer-implemented method of claim 7, further comprising comparing the subsequent epigenetic state to the updated subsequent epigenetic state.

9. The computer-implemented method of claim 8, wherein the updated subsequent epigenetic state is more closely correlated to the experimental epigenetic state than the subsequent epigenetic state that was determined prior to modifying the set of force fields.

10. The computer-implemented method of claim 1, further comprising defining a level of granularity for coarse-grained model.

11. The computer-implemented method of claim 1, wherein one or more functional groups is modeled as one of the particles or a part thereof in the coarse-grained model.

12. The computer-implemented method of claim 1, wherein one or more biomolecules is modeled as one of the particles or a part thereof in the coarse-grained model.

13. The computer-implemented method of claim 1, wherein one or more nucleosomes is modeled as one of the particles or a part thereof in the coarse-grained model.

14. The computer-implemented method of claim 1, wherein each nucleosome of the in silico chromatin complex is represented by one of the particles.

15. The computer-implemented method of claim 1, wherein the external factor is an epigenetic modifying protein, environment chemistry, or physical forces.

16. The computer-implemented method of claim 1, wherein the initial epigenetic state and the subsequent epigenetic state are each based on the presence or absence of an epigenetic tag at a location of the in silico chromatin complex.

17. The computer-implemented method of claim 1, wherein the epigenetic modifying protein is fused to a cas-9 protein.

18. The computer-implemented method of claim 1, wherein the epigenetic modifying protein is a methyltransferase.

19. A system comprising:
one or more data processors; and
a non-transitory computer-readable storage medium comprising instructions which, when executed on the one or more data processors, cause the one or more data processors to perform the computer-implemented method including:
defining an in silico chromatin complex comprising a set of particles, wherein the particles model the in silico chromatin complex as a coarse-grained model;
defining an initial epigenetic state based on the structure and/or composition of the chromatin complex;
defining a set of force fields, wherein each force field independently corresponds to an interaction between at least one of the particles and at least one of: another one of the particles or a nuclear envelope, or the force field corresponds to an interaction between at least one of the particles and the environment surrounding the in silico chromatin complex;
defining a model of an epigenetic modifying process comprising an interaction between the in silico chromatin complex and an external factor, wherein the external factor is capable of changing the initial epigenetic state;
accessing molecular dynamics equations comprising the set of force fields;
iteratively a) solving the molecular dynamics equations for the in silico chromatin complex and b) applying the model of the epigenetic modifying process; and
determining the subsequent epigenetic state resulting from the epigenetic modifying process.

20. A computer-program product tangibly embodied in a non-transitory machine-readable storage medium, comprising instructions configured to cause one or more data processors to perform the computer-implemented method including:
defining an in silico chromatin complex comprising a set of particles, wherein the particles model the in silico chromatin complex as a coarse-grained model;
defining an initial epigenetic state based on the structure and/or composition of the chromatin complex;
defining a set of force fields, wherein each force field independently corresponds to an interaction between at least one of the particles and at least one of: another one of the particles or a nuclear envelope, or the force field corresponds to an interaction between at least one of the particles and the environment surrounding the in silico chromatin complex;
defining a model of an epigenetic modifying process comprising an interaction between the in silico chromatin complex and an external factor, wherein the external factor is capable of changing the initial epigenetic state;
accessing molecular dynamics equations comprising the set of force fields;
iteratively a) solving the molecular dynamics equations for the in silico chromatin complex and b) applying the model of the epigenetic modifying process; and
determining the subsequent epigenetic state resulting from the epigenetic modifying process.
